(19) United States
(12) Patent Application Publication
(10) Pub. No.: US 2015/0112281 A1
(43) Pub. Date: Apr. 23, 2015

(54) SAFETY CANNULA FOR EXTRACORPOEREAL BLOOD TREATMENT

(71) Applicant: Hans HAINDL, (US)

(72) Inventor: Hans Haindl, Wennigsen (DE)

(21) Appl. No.: 14/395,658

(22) PCT Filed: Apr. 19, 2013
(86) PCT No.: PCT/EP2013/058181

§ 371 Date: Oct. 20, 2014

(30) Foreign Application Priority Data
Apr. 20, 2012 (DE) 10 2012 206 557.1

(51) Int. Cl. A61M 25/06; A61M 1/36

(52) U.S. Cl. CPC A61M 25/0631; A61M 1/3659; A61M 25/0637
USPC 604/263

(57) ABSTRACT

The present invention relates to a safety cannula, in particular for extracorporeal blood treatment, comprising a housing and a puncturing cannula having a ground tip, wherein the housing is designed to lie against the skin of a patient during use, and furthermore comprising a safety device which is suitable for automatically pulling the puncturing cannula back into the housing into a protected position when the housing is removed from the skin.

The present application relates to a safety cannula, in particular a safety cannula for extracorporeal blood treatment such as for example for dialysis.

The German Accident Prevention Regulation TRBA 250 stipulates that at all medical workplaces where there is a danger of injury for the user handling cannulas, safety cannulas are to be used, if available, which after use are actively or passively protected such that they no longer pose a danger of injury to the user or third parties. This requirement also applies, inter cilia, to dialysis stations and dialysis centers. During dialysis, a vessel provided artificially under the skin, what is referred to as a shunt, is usually punctured with two cannulas. These cannulas are large bore cannulas and therefore contain relatively large amounts of blood so that in case of an injury of the user by these cannulas, the danger of infection is especially high. In addition, the occurrence of infections transmissible by blood, such as for example Hepatitis B and C as well as HIV, is significantly higher in dialysis patients than the average for the population as a whole.

In order to reduce the risk of infection, so-called safety cannulas have been suggested for dialysis. The principle of these safety cannulas is based on the mechanism that after use of the cannula but before removal of the cannula attachment taped to the patient's skin, the cannula is removed from the blood vessel by pulling on the cannula tube and pulled into the cannula attachment. There, the cannula is locked in place so that the cannula tip cannot re-emerge from the cannula attachment.

Unfortunately, there have been some serious incidents which were due to the fact that the tube on the cannula was not attached separately to the patient's skin by means of a Band-Aid as instructed by the manufacturer. This resulted in tensile forces on the tubing due to, for example, movement by the patient which could lead to the cannula being pulled back into the safety device during dialysis. In the case of the venous cannula, the blood from the dialysis machine was then not returned into the patient's body but into the surroundings. Since in such a case the outflow of the blood is not obstructed, and there is no increase in pressure in the venous lumen, the dialysis machine keeps pumping without sounding an alarm. In the worst case scenario, this could lead to the patient bleeding to death.

The incidents which occurred with safety cannulas illustrate a problem with dialysis which has always existed and to date has not been solved, namely the safety monitoring of the venous lumen of the dialysis. Dialysis machines are equipped with sensitive sensors which detect disruptions in the dialysis procedure and which switch off the machine and/or sound an alarm if necessary. For instance, if the arterial cannula slips out, the cannula will be unable to withdraw any more blood while the cannula tip is still lodged in the tissue, and a negative pressure occurs in the arterial lumen. The dialysis machine registers the malfunction, switches off and sounds an alarm. If the arterial lumen slips out of the tissue completely, it will pull in air instead of blood. This is immediately noticed in the machine, for example by an ultrasound air detector, the machine switches off and sounds an alarm. Thus, the arterial lumen does not pose a safety problem.

The venous lumen is quite another matter. If the venous cannula shifts and its tip lies in the tissue, i.e. between shunt and skin, it is immediately ejected from the tissue by the pumped blood stream, and the blood flows into the surroundings. Usually, the changes in pressure are so small and short-lived that the pressure sensor of the dialysis machine frequently cannot detect them. For this reason, slipped venous cannulas have caused many dialysis patients to bleed to death. Based on the blood flow rates used in dialysis machines, a patient can die after three to five minutes.

The prior art describes various processes and devices by means of which the correct position of a cannula can be monitored, cf. e.g. DE 10 2009 004 018 A1, US 2006/0130591 A1, DE 199 53 068 A1 and WO 99/24145. However, these processes and devices are very complex and furthermore require specific sensor technology. Accordingly, these known processes and devices are expensive and not very practical.

It is therefore the object of the present invention to provide a safety cannula which addresses the above-mentioned safety requirements easily but still reliably. It is another object of the present invention to provide a safety cannula which can be produced at a reasonable cost and which is easy to handle.

These and other objects are achieved by the safety cannula according to claim 1. Additional preferred features are described in the dependent claims.

Accordingly, the present invention provides a safety cannula with a housing and a puncturing cannula with a ground tip. The housing is designed to lie against the skin of a patient. The safety cannula furthermore comprises a safety device which is suitable for automatically pulling the puncturing cannula back into the housing into a protected position when the housing is removed from the skin. In other words, the safety cannula of the invention is designed such that the cannula cannot become detached from the patient with its tip exposed since a removal of the housing from the patient's skin secures the puncturing cannula with the ground tip in the housing. This way, the risk of injury can effectively be minimized.

The present invention is basically significant for all cannulas whose housing is in temporary contact with the skin of a patient. In particular, however, the safety cannula of the present invention is suitable for extracorporeal blood treatment.

For the reasons mentioned above, dialysis is an especially preferred field of application.

In the case of dialysis or other extracorporeal blood treatments, the present invention is based, inter alia, on the fact that due to the automatic retraction of the puncturing cannula into the housing, the flow of blood through the cannula is obstructed. This results in an increase in pressure in the venous inflow which can be registered by the dialysis machine so that it automatically switches off. For this purpose, the safety cannula is preferably designed such that pulling back the puncturing cannula into the protected position obstructs and/or reduces the flow of a liquid through the cannula. It is especially preferred that the flow of a liquid be at least partially prevented. Preferably, this obstruction of the flow of a liquid or blood is achieved by the fact that in the case of the retracted cannula, the sum of the cross-sectional areas of all openings in the housing of the safety cannula is smaller than the cross-sectional area of the cannula so that the retraction of the cannula into the housing generates an increase in pressure in the cannula or its inflow.

Preferably, the housing of the safety cannula comprises an opening at its distal end through which the puncturing cannula extends during use of the cannula so that the ground tip can be introduced into the tissue of the patient. Preferably, this opening has the approximate dimensions of the puncturing cannula. In other words, the inner diameter of this opening essentially corresponds to the outer diameter of the puncturing cannula with a margin of preferably less than 0.5 mm, more preferred less than 0.2 mm and especially preferred less than 0.1 mm. Preferably, the safety device is suitable for covering this distal housing opening at least partially, preferably completely, as soon as the puncturing cannula has been pulled back into the housing. Preferably, the distal housing opening is covered at least partially, preferably essentially completely, by a device in the safety device. This causes a blood congestion when the puncturing cannula is pulled back into the housing which in turn effectively increases the pressure in the puncturing cannula or its inflow.

Preferably, the automatic retraction of the puncturing cannula into a protected position is effected mechanically. According to a preferred embodiment of the invention, the safety device comprises a first spring for automatic retraction, by means of which the puncturing cannula can automatically be pulled back into the housing into a protected position. Preferably, the distal end of the first spring lies flat at the distal end of the housing of the safety cannula and/or is connected to it. The proximal end of the first spring is preferably directly or indirectly connected to the puncturing cannula. During use of the safety cannula, i.e. as long as the tip of the puncturing cannula protrudes from the housing, the first spring is tight or compressed. When the housing of the safety cannula is removed from the patient's skin, the proximal end of the first spring is released so that the spring can relax. This causes the proximal end of the first spring, which is connected to the puncturing cannula, to move in the proximal direction, which pulls the puncturing cannula back into the housing in the proximal direction.

According to the invention, the automatic release of the proximal end of the first spring upon removal of the housing from the patient's skin can be based on different mechanisms. For this purpose, the safety device preferably comprises an element which has been pre-stressed in some manner and which remains in its pre-stressed position when the housing lies against the skin of the patient. However, as soon as the housing becomes detached or is removed from the patient's skin, the pre-stressed element can automatically relax, thus releasing the first spring which in turn can automatically relax as well. Alternatively, however, an element can be provided which blocks the first spring and which slightly adheres to the patient's skin (for example by means of an adhesive strip). When the housing detaches from the patient's skin, the element adhering to the skin moves relatively to the housing so that the blocking of the first spring can be undone.

According to a preferred embodiment of the invention, the safety device comprises a second spring which is suitable for automatically relaxing when the housing is removed from the skin, thus releasing the first spring. In other words, during use of the safety cannula, the second spring is pre-stressed. This pre-stress is maintained by the housing lying against the patient's skin. As soon as the pressure exerted by the skin onto the second spring subsides, the spring can relax and thus release the first spring, or its proximal end. In order to first block the first spring, the second spring preferably comprises a blocking element which can engage, directly or indirectly, with the first spring. The blocking element can be part of the second spring, be integrally connected with it or attached to it. The blocking element can for example comprise one or more locking hooks and/or one or more pins which can directly or indirectly block the first spring. Preferably, the proximal end of the first spring comprises a sleeve with a protrusion with which the locking hooks or pins of the blocking element engage. When the second spring extends, the locking hooks or pins are retracted, thus no longer blocking the sleeve or the first spring which can extend in the proximal direction.

According to a preferred embodiment of the invention, the safety device comprises a covering device suitable for at least partially covering the tip of the cannula and/or the distal opening of the housing during or after the retraction of the puncturing cannula into the housing. Preferably, the covering device is suitable for irreversibly covering the tip of the cannula during or after the retraction of the puncturing cannula into the housing so that the tip of the cannula is no longer accessible and the risk of injury can effectively be minimized. In particular, the covering device preferably prevents a repeated re-emergence of the tip of the cannula from the housing.

Preferably, the covering device comprises a third spring. During use of the safety cannula, the third spring is preferably also pre-stressed and can only relax when the tip of the cannula has been pulled back completely into the housing. The subsequent relaxation of the third spring then causes a cover element, which can be part of the third spring or attached to it, to move in front of the tip of the cannula. The cover element preferably comprises a covering surface area and/or a preferably elastomeric sealing element, by means of which the cannula tip and/or the opening at the distal end of the housing can be at least partially covered to reduce the flow of a liquid through the cannula. Preferably, the opening at the distal end of the housing is at least partially, preferably completely, closed by the covering surface area and/or the sealing element, in order to cause a congestion of the blood flow inside the housing which in turn causes an increase in pressure which can be registered by the dialysis machine. Furthermore, the cover element preferably serves to irreversibly prevent the tip of the cannula from re-emerging from the housing. If the cover element is designed as an elastomeric check valve or unidirectional valve, the irreversible locking of the cannula can preferably be effected at a different location by means of a locking hook.

The first, second and/or third springs can basically be any type of spring or spring mechanism. A compression spring is especially advantageous as a first spring since the puncturing cannula can be fed through the spring. The second and/or third spring is preferably a leaf spring since it can be provided at the inner and/or outer rim of the housing with minimum space requirements. It is especially preferred that the second and third spring form a single-piece leaf spring with two essentially parallel spring arms wherein one fowls the second spring and the other the third spring.

According to a preferred embodiment of the invention, the safety device can be blocked manually in order to prevent an automatic retraction of the puncturing cannula. For this purpose, a portion of the housing can preferably be deformed by finger force so that by pressing together this malleable portion of the housing, the first spring and/or the puncturing cannula can even be fixed if the second spring is extended.

It is furthermore preferred that the cannula be provided such that it can rotate in relation to the housing. Preferably, the rotation of the cannula in relation to the housing can be blocked manually, preferably by pressing together the malleable portion of the housing.

It is furthermore preferred that the safety cannula comprise fold-out wing-type handles which serve, inter alia, to fixate the housing on the skin of the patient. Preferably, the safety device and/or the rotation of the cannula in relation to the housing can be blocked by means of these wing-type handles.

It is furthermore preferred that the design of the housing be telescopic in order to keep the length of the safety cannula as short as possible.

According to a preferred embodiment, the housing of the safety cannula comprises a detachable connection by means of which a release of the safety mechanism prior to the use of the safety cannula can be prevented. Prior to the use of the safety cannula and during introduction of the puncturing cannula into the tissue of the patient, the housing of the safety cannula does not lie against the skin of the patient and is therefore unable, e.g., to hold the second spring in its pre-stressed position. It is therefore preferred that the detachable connection block the safety device, i.e. for example the second spring, until the housing lies against the skin of the patient. Then the detachable connection can be removed in order to activate the safety mechanism. According to a preferred embodiment, an adhesive strip is provided for this purpose which adheres the second spring to the housing. As soon as the housing lies against the skin of the patient, the adhesive strip can be removed in order to activate the safety mechanism. Alternatively, a detachable pin or hook can be provided for this purpose which blocks the second spring and can be removed after the cannula has been placed.

The person skilled in the art will realize that depending on the design of the safety device, an automatic retraction of the puncturing cannula into a protected position upon removal of the housing of the safety cannula from the skin can only be effected at a certain minimum distance between housing and skin. Therefore, within the framework of the present invention, the term “removing” has to be understood such that in the area of the safety device (i.e. in the case of the preferred embodiment in the area of the second spring), the housing is removed from the skin of the patient such that the corresponding safety device is activated. Preferably, this takes place at a distance of at least 2 mm, more preferred at least 4 mm and especially preferred at least 5 mm.

Preferably, the housing comprises a fitting for a tube, or is integrally connected with a tube.

The safety cannula according to the invention can be produced easily and at a reasonable cost, guarantees a high degree of safety against injuries and solves the problems associated with dialysis cannulas mentioned above.

Preferred embodiments of the safety cannula according to the invention are described in detail below with reference to the Figures.

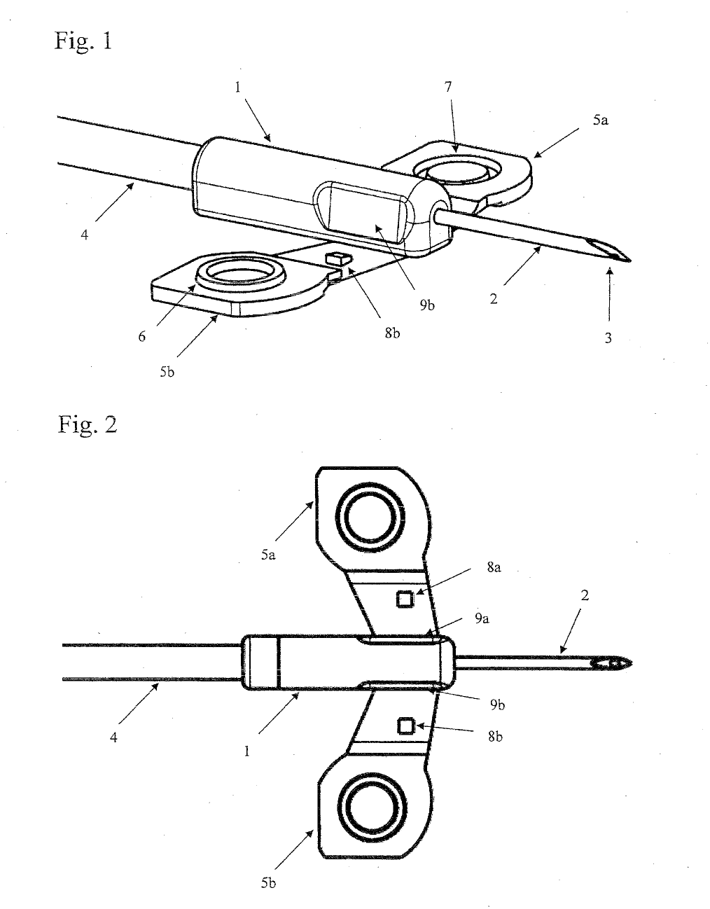

FIG. 1 shows a perspective view of a preferred embodiment of the safety cannula according to the invention;

FIG. 2 shows a top view of the safety cannula of FIG. 1;

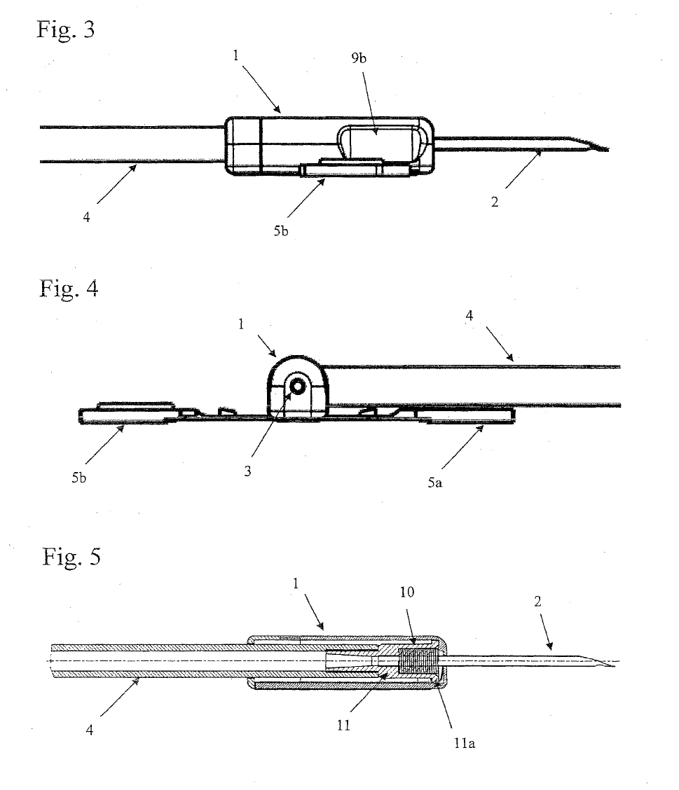

FIG. 3 shows a side view of the safety cannula of FIG. 1;

FIG. 4 shows a front view of the safety cannula of FIG. 1;

FIG. 5 shows a side sectional view of the safety cannula of FIG. 1;

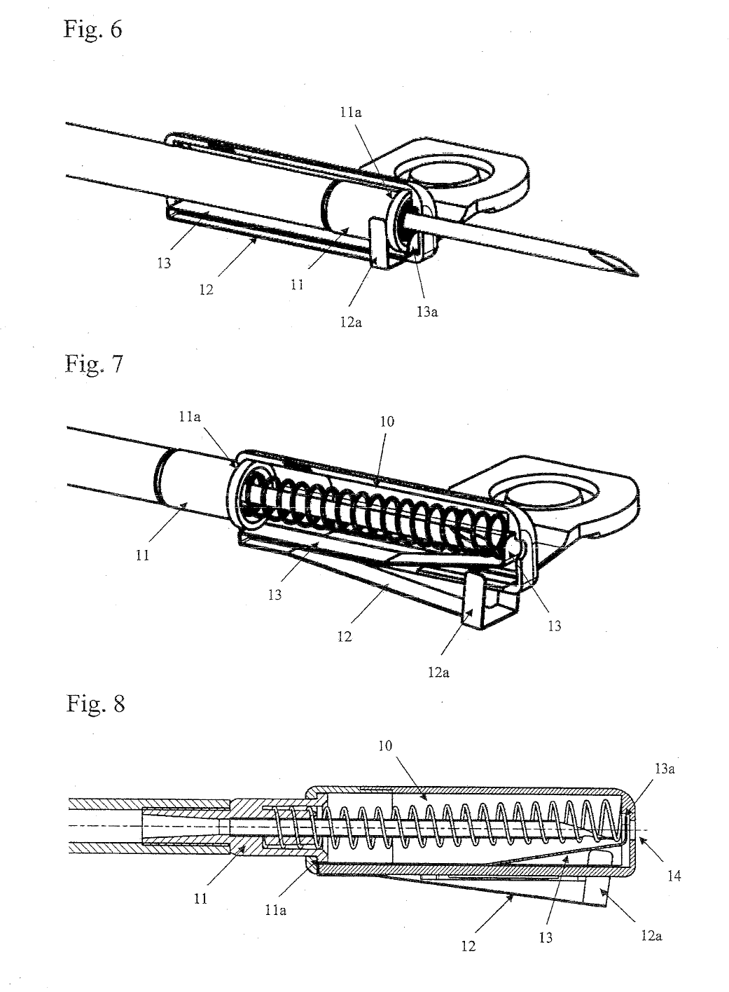

FIG. 6 shows a perspective sectional view of the safety cannula of FIG. 1;

FIG. 7 shows a perspective view of the safety cannula of FIG. 1 with a retracted tip of the cannula;

FIG. 8 shows a side sectional view of the safety cannula of FIG. 1 with a retracted tip of the cannula;

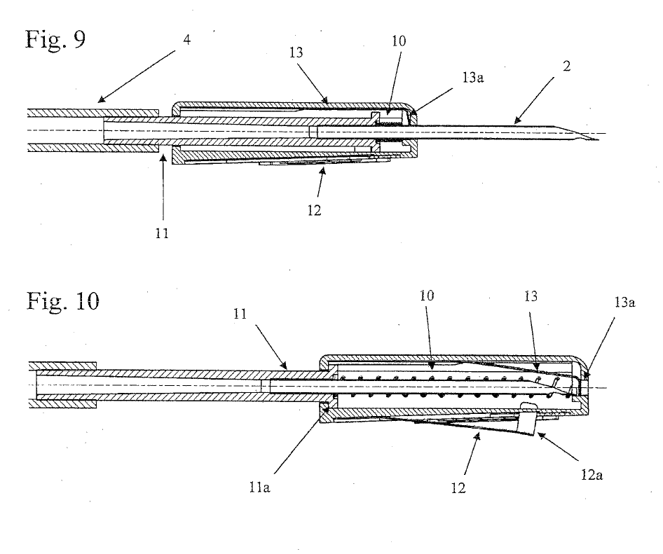

FIG. 9 shows a side sectional view of another preferred embodiment of the safety cannula according to the invention; and

FIG. 10 shows a side sectional view of the safety cannula of FIG. 9 with a retracted tip of the cannula.

FIGS. 1 to 4 show a preferred embodiment of the safety cannula according to the invention in a perspective view, a top view, a side view and a front view. The safety cannula comprises a housing 1, a puncturing cannula 2 with a ground tip 3, a tube 4 and two wing-type handles 5a and 5b. The two wing-type handles comprise a circular protrusion 6 and/or a circular groove 7 which can mesh when the wing-type handles are folded up. The housing 1 comprises two opposing malleable housing portions 9a and 9b whose housing wall is designed to be especially thin and/or which consist of a soft material, so that these housing portions can easily be deformed by finger force. When the two wing-type handles 5a and 5b are folded up and mesh via the circular components 6 and 7, the two protrusions 8a and 8b on the wing-type handles 5a and 5b press into the malleable housing portions 9a and 9b, thus blocking the safety device.

The safety device can be seen in the sectional views of FIGS. 5 to 8. The safety device comprises, inter alia, a first spring 10 for automatically pulling back the puncturing cannula 2 by means of which the puncturing cannula 2 can be pulled back into a protected position in the housing from the position shown in FIGS. 5 and 6, as is shown in FIGS. 7 and 8. During use of the safety cannula, the first spring 10 is pre-stressed, or compressed, as is shown in FIG. 5. The distal end of the first spring 10 lies flat at the distal end of the housing 1 and/or is connected to it. The proximal end of the first spring 10 is connected with a sleeve 11. Furthermore, the proximal end of the first spring 10 and/or the sleeve 11 is/are connected with the puncturing cannula 2 so that an extension of the first spring 10 causes the sleeve 11 and the puncturing cannula 2 to move in the proximal direction from the position shown in FIG. 5 to the position shown in FIGS. 7 and 8.

The safety device furthermore comprises a second spring 12 and a third spring 13 which are formed as a single-piece leaf spring with two arms as shown in the preferred embodiment. At its distal end, the second spring 12 comprises two locking hooks 12a which mesh with a circular protrusion 11a at the distal end of the sleeve 11 (cf. FIG. 6) so that the sleeve 11 is fixed in its distal position shown in FIGS. 5 and 6 and the first spring 10 is blocked in its compressed position. Since during use of the safety cannula the housing lies against the skin of a patient, the second spring 12 is pressed to the wall of the housing against its spring force so that the locking hooks 12a mesh with the circular protrusion 11a. When the housing is removed from the patient's skin, the second spring 12 can spring back from the housing wall, as is shown in FIGS. 7 and 8, so that the locking hooks 12a slide out from behind the circular protrusion 11a of the sleeve 11, thus releasing the sleeve 11 or the first spring 10. This allows the first spring 10 to extend automatically so that its proximal end as well as the sleeve 11 are moved in the proximal direction together with the puncturing cannula 2 until the tip 3 of the cannula has been pulled back completely into the housing 1 of the safety cannula.

As soon as the tip 3 of the cannula has moved past the cover element 13a provided at the third spring 13, the spring 13 extends such that the cover element 13a slides between the tip 3 of the cannula and a distal opening 14 of the housing as is shown in FIGS. 7 and 8. Thus, the cover element 13a prevents the cannula 2 from re-emerging from the housing 1 and at the same time closes the distal opening 14 of the housing which leads to a congestion of the blood flow inside the housing 1. Even if the distal opening 14 of the housing is not completely closed by the cover element 13a, the blood flow is obstructed by the cover element 13a so that within the cannula 2 or within the tube 4, an increase in pressure takes place which can be registered by a dialysis machine connected to the cannula.

FIGS. 9 and 10 show another preferred embodiment of the safety cannula according to the invention in a sectional view both with an extended tip of the cannula (FIG. 9) and in a protected position (FIG. 10). While the second spring 12 and the third spring 13 are provided as a single-piece leaf spring with two arms in the preferred embodiment shown in FIGS. 1 to 8, the preferred embodiment shown in FIGS. 9 and 10 shows two separate second and third springs 12 and 13 which are not formed as a single piece or integrally. Rather, the second spring 12 is provided in the form of a leaf spring below the cannula 2 or the first spring 10, and the third spring 13 is provided in the form of another leaf spring above the cannula 2 or the first spring 10. In the case of the second and/or third springs, the leaf spring can also be replaced with another type of spring. Alternatively, the second spring 12 can also be provided above the cannula 2 or the first spring 10, and the third spring 13 can also be provided below the cannula 2 or the first spring 10.

This embodiment, which essentially works the same way as the first embodiment, has the advantage that the spring constants and/or the reaction times of the second and third springs can easily be adjusted independently of each other. Furthermore, the area of this embodiment between the tissue of the patient and the cannula can be designed to be narrower.

1-18. (canceled)

19. Safety cannula, in particular for extracorporeal blood treatment, comprising a housing and a puncturing cannula having a ground tip, wherein the housing is designed to lie against the skin of a patient during use, and furthermore comprising a safety device which is suitable for automatically pulling the puncturing cannula back into the housing into a protected position when the housing is removed from the skin.

20. Safety cannula according to claim 19, wherein the safety device comprises a first spring for automatic retraction, by means of which the puncturing cannula can automatically be pulled back into the housing into a protected position.

21. Safety cannula according to claim 19, wherein the safety device comprises a second spring which automatically relaxes when the housing is removed from the skin, thus releasing the first spring.

22. Safety cannula according to claim 19, wherein the safety device comprises a covering device suitable for at least partially covering the tip of the cannula and/or an opening at the distal end of the housing when the puncturing cannula is pulled back into the housing.

23. Safety cannula according to claim 22, wherein the covering device irreversibly covers the tip of the cannula when the puncturing cannula is pulled back into the housing.

24. Safety cannula according to claim 22, wherein the covering device comprises a third spring.

25. Safety cannula according to claim 24 wherein the second and/or third spring comprises a leaf spring.

26. Safety cannula according to claim 24, wherein the second and third springs form a single-piece leaf spring.

27. Safety cannula according to claim 24, wherein the second and third springs each form a separate leaf spring and wherein the second and third springs are provided at opposite ends of the cannula.

28. Safety cannula according to any of claim 21, wherein the second spring comprises one or more locking hooks which directly or indirectly engages with the first spring.

29. Safety cannula according to claim 28, wherein the proximal end of the first spring comprises a sleeve with which the locking hooks of the second spring engages.

30. Safety cannula according to claim 22, wherein the covering device comprises a covering surface area and/or an elastomeric sealing element by means of which the cannula tip and/or an opening at the distal end of the housing can be at least partially covered to reduce, preferably at least partially prevent, the flow of a liquid through the cannula.

31. Safety cannula according to claim 19, wherein the safety device can be blocked manually in order to prevent an automatic retraction of the puncturing cannula.

32. Safety cannula according to claim 31, wherein the blocking is carried out by pressing together a malleable portion of the housing.

33. Safety cannula according to claim 19, wherein the safety cannula comprises wing-type handles by means of which the safety device can be blocked.

34. Safety cannula according to any of the preceding claims, wherein the design of the housing is telescopic.

35. Safety cannula according to claim 19, wherein the automatic retraction of the cannula takes place at a distance of at least 2 mm, preferably at least 4 mm and especially preferred at least 5 mm between the housing and the skin of the patient.

36. Safety cannula according to claim 19, wherein a detachable connection, preferably an adhesive strip, is provided at the housing in order to prevent a release of the safety mechanism prior to the use of the safety cannula.
